(19) United States
(12) Patent Application Publication
(10) Pub. No.: US 2019/0185428 A1
(43) Pub. Date: Jun. 20, 2019

(54) METHOD FOR PRODUCING CHIRAL AMINONITRILES

(71) Applicant: UNIVERSITÄT BIELEFELD, Bielefeld (DE)

(72) Inventors: Harald GRÖGER, Bielefeld (DE); Tobias BETKE, Bielefeld (DE); Philipp ROMMELMANN, Bielefeld (DE)

(73) Assignee: UNIVERSITÄT BIELEFELD, Bielefeld (DE)

(21) Appl. No.: 16/328,519

(22) PCT Filed: Aug. 17, 2017
(86) PCT No.: PCT/EP2017/070820

§ 371 Date: Feb. 26, 2019

(30) Foreign Application Priority Data
Aug. 30, 2016 (DE) 10 2016 116 130.6

(51) Int. Cl. C07D 207/16; C07B 51/00

(52) U.S. Cl. CPC C07D 207/16; C07B 2200/07; C07B 51/00

(57) ABSTRACT

The invention relates to a method for preparing an N-acyl- or N-sulfonyl-α-aminonitrile, comprising the following steps: a) condensation of an N-acyl- or N-sulfonyl-α-aminoaldehyde with hydroxylamine to give an aldoxime, and b) dehydration of the aldoxime obtained in step a) to give an N-acyl- or N-sulfonyl-α-aminonitrile. In an advantageous manner, the absolute configuration can be retained in the conversion to the N-acyl- or N-sulfonyl-α-aminonitrile.

The invention relates to the field of organic synthesis, in particular a method for preparing chiral N-acyl- and N-sulfonyl-α-aminonitriles.

Enantiomerically enriched, especially enantiomerically pure, N-acyl-α-aminonitriles of the (R) and (S) type are valuable synthesis units in the production of modern medicaments having a chiral nitrile unit, or constitute such medicaments. Examples of such active pharmaceutical ingredients are gliptins such as vildagliptin and saxagliptin, and also NVP-DPP-728. Gliptins act as dipeptidyl peptidase-4 inhibitors and are used as medicaments for treating type 2 diabetes mellitus. The active ingredient vildagliptin was developed by Novartis and marketed in 2013 for type 2 diabetes with a sales volume of 1.2 billion US dollars. A method for the production thereof is described in the document WO 2000 034 241 A. Saxagliptin and a method for the production thereof is described in the document WO 2004 052 850 A.

Enantiomerically pure N-protected or N-acylated pyrrolidine-2-nitrile derivatives are an important intermediate in the synthesis of these gliptins. Typically, N-acylated chiral nitriles of the (R) and (S) type are still accessed by multi-stage syntheses. A disadvantage of the known synthetic approaches to enantiomerically pure N-acyl-α-aminonitriles in the prior art is particularly that these are based on the use of highly toxic cyanides or other toxic reagents such as the Vilsmeier reagent. In their preparation, already toxic reagents such as oxalyl chloride or phosphorus oxychloride are also used.

For instance, the preparation of α-aminonitriles, which are readily accessible via the Strecker reaction, which is the most known method for preparing chiral enantiomerically enriched or enatiomerically pure nitriles, is based on the use of highly toxic cyanides. To stabilize these generally rather labile compounds which also have a tendency to the reverse reaction releasing highly toxic hydrogen cyanide, these are preferably acylated. However, these syntheses are typically carried out using acyclic imines, which neither achieves a direct synthetic approach to proline-analogous nitriles nor to α-aminonitriles having a primary amino group as nitrile analogues of the acyclic proteinogenic α-amino acids. From the perspective of chemical and process safety and also the sustainability and environmental compatibility of a chemical production process, cyanide-free routes to nitriles are of major interest.

Derivatization methods starting from enantiomerically pure amino acids are a known and industrially applied alternative for producing nitriles derived from amino acids. In this case, the amino acid is firstly converted to an amide before this amide is subsequently activated and converted to the desired nitrile. This synthetic approach, which is based on the use of a Vilsmeier reagent and on the concept of “chiral pool derivatization”, is used for example in the synthesis of vildagliptin, as described by L. Pellegatti and J. Sedelmeier in Org. Process Res. Dev., 2015, 19, pp. 551-554. A disadvantage of this process, however, is that firstly the amide has to be synthesized in a laborious manner from L-proline. Even the preparation of unsubstituted amides using only ammonia is not trivial. Secondly, the so-called “Vilsmeier reagent” must be prepared in a laborious manner and is associated with a high amount of waste. A need therefore exists for alternative synthetic methods for chiral α-aminonitriles.

Therefore, the object of the present invention was to provide a method that overcomes at least one of the aforementioned disadvantages of the prior art. In particular, the object of the present invention was to provide a method which allows the preparation of chiral α-aminonitriles independently of highly toxic cyanides and problematic reagents such as the Vilsmeier reagent.

This object is achieved by a method for preparing an N-acyl- or N-sulfonyl-α-aminonitrile, comprising the following steps:
a) condensation of an N-acyl- or N-sulfonyl-α-aminoaldehyde with hydroxylamine to give an aldoxime, and
b) dehydration of the aldoxime obtained in step a) to give an N-acyl- or N-sulfonyl-α-aminonitrile.

The method according to the invention allows the preparation of chiral N-acyl- or N-sulfonyl-α-aminonitriles in a preparatively simple and economical manner, starting from readily accessible N-acyl-α-aminoaldehydes or N-sulfonyl-α-aminoaldehydes as substrate component and the conversion of the aldehyde component to an aldoxime unit via condensation with hydroxylamine and subsequent dehydration of the aldoxime unit to give the nitrile. Surprisingly it has been found that during this synthetic process, the enantiomeric purity is retained or is only reduced by a negligible amount and thus the racemization via keto-enol tautomerization typically observed in such compound classes capable of enol formation can be suppressed. The method is particularly suitable for the preparation of enantiomerically enriched and preferably enantiomerically pure N-acyl- or N-sulfonyl-α-aminonitriles.

The method uses N-acyl- or N-sulfonyl-α-aminoaldehydes as reactant, which are readily obtainable in an advantageous manner in enantiomerically enriched, especially enantiomerically pure form, starting from α-amino acids by N-acylation and conversion of the carboxylic acid function to an aldehyde function. On account thereof, the method provides many advantages. Based on readily accessible amino acids and hydroxylamine as bulk chemicals, aldoximes are readily accessible as substrate. Furthermore, the reaction steps are robust with respect to racemization. Of further advantage is that the method does not require the use of highly toxic cyanide or Vilsmeier reagent that is laborious to synthesize and is associated with considerable amounts of waste. The method can be carried out easily on a preparative scale and is characterized by high practicability. Overall, the method allows in an advantageous manner the preparation of the desired N-acyl- and N-sulfonyl-α-aminonitriles, starting from readily accessible and cost-effective starting compounds, under mild conditions without using problematic reagents. In addition, high conversions, high yields and excellent enantiomeric excesses are achievable.

The method is suitable for preparing chiral N-acyl- and N-sulfonyl-α-aminonitriles. In the context of the present invention, the term “chiral” is understood to mean a compound having at least one stereocentre, the substituents of which cannot change their position relative to one another. As a result, different spatial arrangements are possible. This is the case, for example, if a carbon atom in a molecule bears four different substituents. This carbon atom is referred to as a stereocentre or chiral centre. In preferred embodiments, an enantiomerically enriched or enantiomerically pure N-acyl- or N-sulfonyl-α-aminoaldehyde is used in step a). A major advantage of the method is that its absolute configuration is retained or substantially retained in the conversion to the N-acyl- or N-sulfonyl-α-aminonitrile. The expression that the absolute configuration is substantially retained is understood to mean that the enantiomeric excess, or ee for short, may easily diminish, for example from ≥99% ee to ≥95% or ≥90% ee.

In preferred embodiments, the method comprises the following steps:
a) condensation of an N-acyl- or N-sulfonyl-α-aminoaldehyde according to the general formula (I) with hydroxylamine to give an aldoxime according to the general formula (II), and
b) dehydration of the aldoxime obtained in step a) to give an N-acyl- or N-sulfonyl-α-aminonitrile according to the general formula (III):

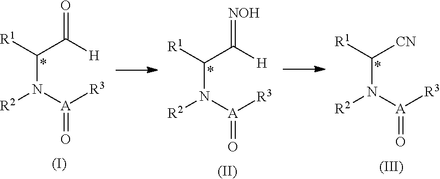

in which:
A is C or S═O;
R¹ is selected from the group comprising branched or unbranched C₁-C₂₀-alkyl, C₆-C₁₀-aryl, C₆-C₁₆-heteroaryl, C₇-C₁₆-arylalkyl and/or C₆-C₁₆-heteroarylalkyl, wherein these are unsubstituted or monosubstituted or polysubstituted by at least one substituent selected from the group comprising OH, NH₂, NHR⁴, NR⁴₂, C₁₋₄-alkyl, C₇-C₁₆-arylalkyl, C₆-C₁₆-heteroarylalkyl, carbonyl oxygen and/or C₁₋₄-alkoxy;
R² is selected from the group comprising H, branched or unbranched C₁-C₂₀-alkyl, C₆-C₁₀-aryl, C₆-C₁₆-heteroaryl, C₇-C₁₆-arylalkyl and/or C₆-C₁₆-heteroarylalkyl, wherein these are unsubstituted or monosubstituted or polysubstituted by at least one substituent selected from the group comprising OH, NH₂, NHR⁴, NR⁴₂, C₁₋₄-alkyl, C₇-C₁₆-arylalkyl, C₆-C₁₆-heteroarylalkyl, carbonyl oxygen and/or C₁₋₄-alkoxy; or
R¹ and R² together form a saturated 5- or 6-membered ring or a bicyclic ring system, wherein these may comprise at least one further heteroatom selected from N, O and/or S and/or these can be monosubstituted or polysubstituted by at least one substituent selected from the group comprising OH, NH₂, NHR⁴, NR⁴₂, C₁₋₄-alkyl, carbonyl oxygen and/or C₁₋₄-alkoxy;
R³ is selected from the group comprising H, C₁₋₆-alkoxy and/or C₁₋₆-alkyl, wherein these are monosubstituted or polysubstituted by at least one substituent selected from the group comprising OH, OR⁴, NH₂, NHR⁴, NR⁴₂, NHY and/or halogen; or is selected from the group of the structural elements (IV), (V) and (VI) as follows:

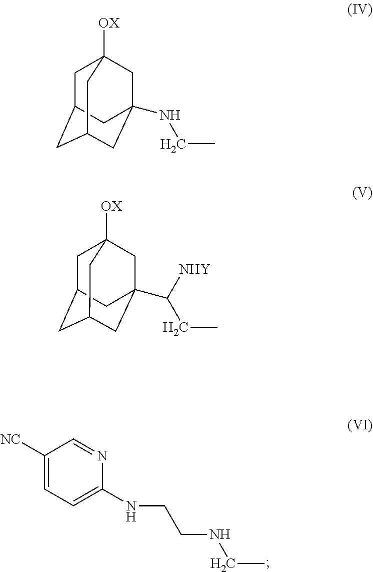

R⁴ is in each case identical or each independently selected from the group comprising C₁-C₁₈-alkyl or C₁-C₁₈-acyl;
X, Y are in each case identical or each independently H or a protecting group, especially selected from tert-butyloxycarbonyl, benzyloxycarbonyl, acetyl, silyl, p-tolyl, trifluoromethyl and/or sulfonyl.

The term “C₁-C₂₀-alkyl” includes, unless stated otherwise, straight-chain or branched alkyl groups having 1 to 20 carbon atoms. Alkyl groups are preferably selected from the group comprising methyl, ethyl, propyl, isopropyl, butyl, isobutyl, pentyl, isopentyl, neopentyl, hexyl, isohexyl, heptyl, isoheptyl, octyl, isooctyl, 2-ethylhexyl, neooctyl, nonyl, isononyl, neononyl, decyl, isodecyl and/or neodecyl. Preference is given to C₁-C₆-alkyl groups selected from the group comprising methyl, ethyl, propyl, isopropyl, butyl and/or tert-butyl.

The term “aryl” is understood to mean aromatic radicals having 6 to 10 carbon atoms. The term “aryl” includes preferably carbocycles, especially phenyl.

In the context of the present invention, the term “arylalkyl” is understood to mean that this is bonded via the alkyl moiety. The aryl moiety may comprise 6 to 10 carbon atoms and the alkyl moiety 1 to 6 carbon atoms, preference being given to phenylalkyl having 1 to 4 carbon atoms in the alkyl moiety, especially benzyl.

C₁-C₆-alkoxy groups are preferably selected from the group comprising methoxy, ethoxy, linear or branched propoxy and/or butoxy.

In the context of the present invention, unless stated otherwise, the term “heteroaryl” is understood to mean mono-, bi- or tricyclic heteroaryl groups comprising one, two, three or four heteroatoms selected from the group comprising N, O and/or S. Preferred heteroaryl groups are monocyclic heteroaryl groups. Preferred monocyclic heteroaryl groups comprise one heteroatom. Preferred heterocyclyl groups are selected from the group comprising furanyl, pyrrolyl, pyridinyl and/or thienyl. Particularly preferred heteroaryl groups are selected from the group comprising furanyl and/or thienyl.

In the context of the invention, the term “C₁-C₁₈-acyl” includes preferably straight-chain or branched acyl groups having 1 to 18 carbon atoms. Preferred C₁-C₁₀-acyl groups are selected from the group comprising formyl, acetyl, propanoyl, isopropanoyl, butanoyl, isobutanoyl, pentanoyl and/or isopentanoyl. Preference is given to a straight-chain or branched C₁-C₄-acyl radical. Particular preference is given to acetyl.

The term “halogen” includes fluorine, chlorine, bromine and iodine, wherein fluorine or chlorine, especially chlorine, is preferred.

In the context of the invention, the term “protecting group” describes a substituent which is introduced during the synthesis in order to temporarily protect a functional group, for example a hydroxyl group, and to prevent undesired reactions. In the context of the method, the protecting group can be cleaved again or remain on the N-acyl- or N-sulfonyl-α-aminonitrile, for example if this is intended to be used for further synthetic steps. Preferred protecting groups are selected from tert-butyloxycarbonyl (Boc), benzyloxycarbonyl, acetyl, silyl, p-tolyl, trifluoromethyl and/or sulfonyl. Preferred silyl protecting groups are selected from trimethylsilyl (TMS), tert-butyldimethylsilyl (TBDMS), triethylsilyl (TES), tert-butyldiphenylsilyl (TBDPS) and triisopropylsilyl (TIPS). Preferred sulfonyl protecting groups are selected from p-toluenesulfonate (tosyl) or methylsulfonate (mesyl). Protecting groups may be used in particular to obtain N-protected or N-acylated pyrrolidine-2-nitrile derivatives, which can be used advantageously in syntheses of the gliptins.

In preferred embodiments, A is a carbon atom. In particular, N-acyl-α-aminonitriles can be used in an advantageous manner as synthesis units for medicaments having chiral nitrile units or to form an active ingredient. It can also be preferred that A is an S═O group. Chiral N-sulfonyl-α-aminonitriles can also be used advantageously in active ingredient chemistry. In particular, a sulfonyl group can be readily cleaved such that a primary or secondary amino group can be made available.

The substituents R¹ and R² can be identical or each independently branched or unbranched C₁-C₅-alkyl, phenyl or C₇-C₁₀-phenylalkyl. The substituent R² may also be hydrogen in this case. In other embodiments, the substituents R¹ and R² may together form a saturated 5- or 6-membered ring or a bicyclic ring system. The ring system formed already comprises a nitrogen atom in these cases, but may also comprise further heteroatoms, particularly nitrogen or oxygen. The substituents R¹ and R² may each in turn also be substituted, particularly by a group selected from OH, NH₂, C₁₋₄-alkyl or a carbonyl oxygen.

The substituent R³ may be hydrogen, especially in the case that A is a carbon atom. Preferably, protecting groups typically applied for the amino function of amino acids are used. Particularly for the case that A is a carbon atom, the substituent R³ is preferably a C₁₋₅-alkoxy group, particularly tert-butoxy, or a halogen-substituted, especially chlorine-substituted C₁₋₃-alkyl group, especially chloromethyl. Particularly in the context of the synthesis of the gliptins, the substituent R³ is a structural element (IV), (V) or (VI). Particularly in the synthesis of the gliptins, preference is given to enantiomerically pure N-protected or N-acylated pyrrolidine-2-nitrile derivatives as product of the method.

N-acyl- or N-sulfonyl-α-aminoaldehydes that can be used as substrate are commercially available or are readily obtainable, for example starting from α-amino acids, by N-acylation and conversion of the carboxylic acid function to an aldehyde function. The substituents R¹ and R² in embodiments can therefore correspond to the side chains of amino acids. In particular, phenylalanine and proline can be used advantageously as amino acids. In an especially preferred embodiment, R¹ can be benzyl while R² is hydrogen. In this case, the substrate can be provided starting from the amino acid phenylalanine. In a further especially preferred embodiment, R¹ and R² can together form a saturated 5-membered ring. In this case, the substrate can be provided starting from the amino acid proline. L-proline is a readily accessible natural substance.

In a preferred embodiment, the substituents of the compounds according to the general formulae (I), (II) and (III) are the following:
A is C;
R¹ is selected from the group comprising benzyl and/or C₁-C₂-alkyl,
R² is selected from the group comprising H, benzyl and/or C₁-C₂-alkyl, or
R¹ and R² together form a saturated 5-membered ring or bicyclo[3.1.0]hexane, and
R³ is selected from the group comprising H, tert-butoxy, chloromethyl, structural elements (IV), (V) and/or (VI).

In an advantageous manner, the reaction steps are robust against racemization. For instance, chiral N-acyl- or N-sulfonyl-α-aminonitriles with excellent enantiomeric excess can be achieved in enantiomerically enriched, especially enantiomerically pure form.

Further advantages arise therefrom in that the method allows the preparation of N-acyl- or N-sulfonyl-α-aminonitrile in a preparatively simple form and under mild conditions. As a result, the method can be carried out preparatively in a simple and economically viable manner. In addition, high conversions and high yields of enantiomerically enriched or enantiomerically pure product can be achieved.

The dehydration in step b) is preferably effected using a chemocatalyst. In preferred embodiments, the dehydration of the aldoxime to give the N-acyl- or N-sulfonyl-α-aminonitrile in step b) is carried out in the presence of a transition metal catalyst, especially a Cu(II), Zn(II), Co(II) or Ni(II) catalyst. Cu(II)-based chemocatalysts have proven to be particularly suitable for this purpose. Particular preference is given to copper(II) acetate.

In preferred embodiments, the mole fraction of the catalyst is in the range from ≥0.1 mol % to ≤25 mol %, preferably in the range from ≥1 mol % to ≤10 mol %, preferably in the range from ≥2 mol % to ≤5 mol %. The mole fraction of the catalyst in this context is based on the amount of substrate. In particular, good results were achieved at amounts used of just 2 mol % Cu(II) as catalytically active metal species.

Preferably, the condensation of the aldehyde, especially according to the general formula (I), in step a) with hydroxylamine is carried out in aqueous solution, especially in a mixture of water and alcohol. Preferred alcohols are selected from the group comprising methanol, ethanol, isopropanol, n-propanol, n-butanol, tert-butanol, phenol and/or mixtures thereof. The alcohol is selected in particular from n-propanol and/or ethanol. Particularly suitable are mixtures of water and alcohol, for example mixtures of water with ethanol and/or n-propanol. The aldoxime can be isolated and purified from aqueous or alcoholic solution in a simple manner.

For the dehydration of the aldoxime to give the nitrile in step b), organic solvents in particular can be used. The dehydration of the aldoxime to give the N-acyl- or N-sulfonyl-α-aminonitrile is preferably carried out in a solvent selected from dichloromethane, methyl tert-butyl ether, ethyl acetate, tetrahydrofuran, 2-methyltetrahydrofuran, toluene, acetonitrile, propionitrile, butyronitrile and/or mixtures thereof. In preferred embodiments, the dehydration of the aldoxime to give the N-acyl- or N-sulfonyl-α-aminonitrile in step b) is carried out in the presence of a nitrile component. The nitrile is preferably selected from the group comprising acetonitrile, propionitrile and/or butyronitrile. Particular preference is given to acetonitrile. In an advantageous manner, these nitriles form a good and selective reagent for the conversion of the aldoxime to the corresponding nitriles by dehydration. The nitrile component is preferably present in molar excess, for example in the range of ≥10 eq. (equivalents) based on the aldoxime. In this way, rearrangement to the amide can be prevented or significantly suppressed. The molar ratio of acetonitrile to aldoxime is preferably at least 10:1. The nitrile component may also be present at a higher proportion, for example ≥15 eq., based on the aldoxime. In further embodiments, preference is given to mixtures of a nitrile, especially acetonitrile, with other solvents such as dichloromethane.

The condensation of the aldehyde with hydroxylamine in step a) can be carried out at ambient temperature. The dehydration of the aldoxime to give the nitrile in step b) is preferably carried out at elevated temperatures or under reflux. In preferred embodiments, the dehydration of the aldoxime to give the N-acyl- or N-sulfonyl-α-aminonitrile in step b) is conducted at a temperature in the range from ≥20° C. to ≤150° C., preferably in the range from ≥50° C. to ≤100° C., preferably in the range from ≥80° C. to ≤85° C. The fact that the reaction can be carried out at mild temperatures significantly simplifies the reaction regime. It can be envisaged that a reaction time of 1 to 7 or 8 hours at these temperatures is followed by a further reaction phase of up to 20 hours at ambient temperature.

Of particular advantage is that the method can simplify the synthesis of the gliptins suitable as active pharmaceutical ingredient. For instance, the method is particularly suitable for preparing enantiomerically pure N-protected pyrrolidine-2-nitrile, for example the N-Boc-protected analogues. This compound type, which can also be regarded as cyano analogues of N-acylated L-proline, represents an important intermediate in the production of the active ingredient vildagliptin and also the active ingredient NVP-DPP-728. The method is also advantageously suitable for the preparation of N-acylated pyrrolidine-2-nitrile derivatives, which are suitable as intermediates for the production of saxagliptin.

A particular aspect of the invention relates to a method for preparing vildagliptin or salts thereof, comprising the following steps:
a) condensing an aldehyde of the formula (1) with hydroxylamine to give an aldoxime of the formula (2):

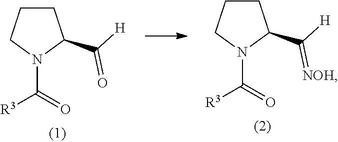

in which
R³ is —CH₂— substituted by a substituent selected from the group comprising OH, OR⁴, NH₂, NHR⁴, NR⁴₂, NHY and/or halogen or structural element (IV) as follows:

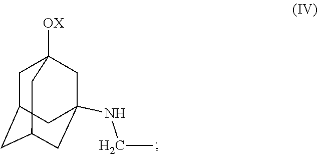

X, Y are identical or each independently H or a protecting group, especially selected from tert-butyloxycarbonyl (Boc), benzyloxycarbonyl, acetyl, silyl, p-tolyl, trifluoromethyl and/or sulfonyl:
R⁴ is identical or each independently selected from the group comprising C₁-C₁₈-alkyl or C₁-C₁₈-acyl;
b) dehydration of the aldoxime of the formula (2) obtained in step a) to give an N-acyl-α-aminonitrile of the formula (3):

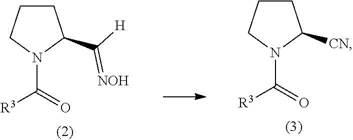

c) optional reaction of the N-acyl-α-aminonitrile of the formula (3), where R³ is —CH₂— substituted by a substituent selected from the group comprising OH, OR⁴, NH₂, NHR⁴, NR⁴₂, NHY and/or halogen, with 1-aminoadamantan-3-ol or a protected derivative of the formula (4) to give the compound of the formula (5):

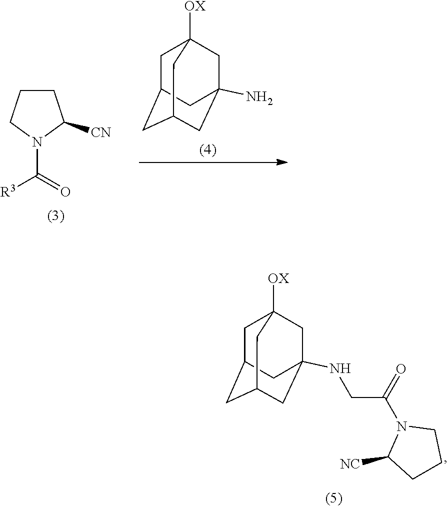

and
d) optional cleavage of the protecting group X to give vildagliptin of the formula (6):

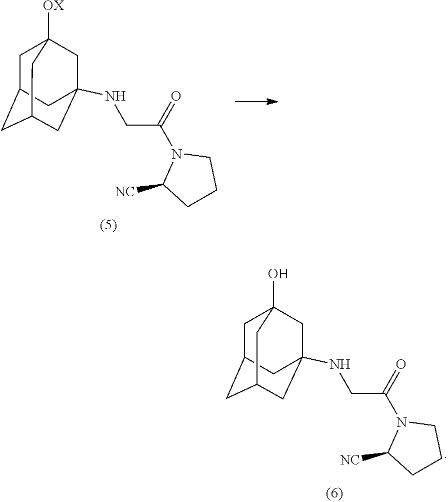

The introduction of the adamantyl radical in the production of vildagliptin can be carried out in a step downstream of the preparation of the nitrile or alternatively can already be present at the oxime stage. Accordingly, the substituent R³ in the aldehyde (1) can be a substituted —CH₂— group or the adamantyl element (IV). In the case that the adamantyl radical is already present in the aldehyde (1), the N-acyl-α-aminonitrile (3) already corresponds to the desired, optionally protected, vildagliptin and step c) can be omitted.

The substituents X and Y are each hydrogen or a protecting group. In particular, preference is given to readily cleavable acyl protecting groups such as tert-butyloxycarbonyl (Boc), acetyl or silyl, in particularly trimethylsilyl or —S(O₂)R, especially tosyl (CH₃—C₆H₄—SO₂—).

A further particular aspect of the invention relates to a method for preparing saxagliptin or salts thereof, comprising the following steps:
a) condensation of an aldehyde of the formula (7) with hydroxylamine to give an aldoxime of the formula (8):

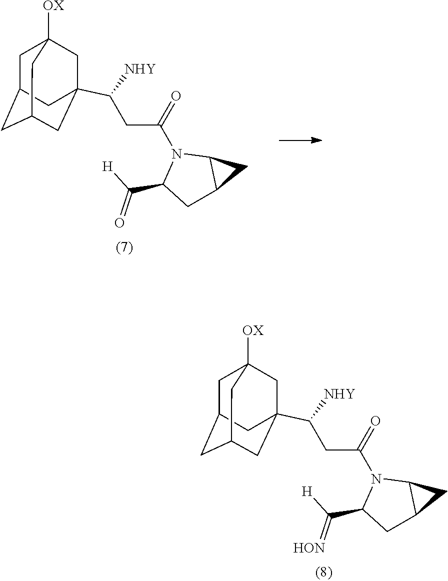

in which:
X, Y are identical or each independently H or a protecting group, especially selected from tert-butyloxycarbonyl (Boc), benzyloxycarbonyl, acetyl, silyl, p-tolyl, trifluoromethyl and/or sulfonyl;
b) dehydration of the aldoxime of the formula (8) obtained in step a) to give an N-acyl-α-aminonitrile of the formula (9):

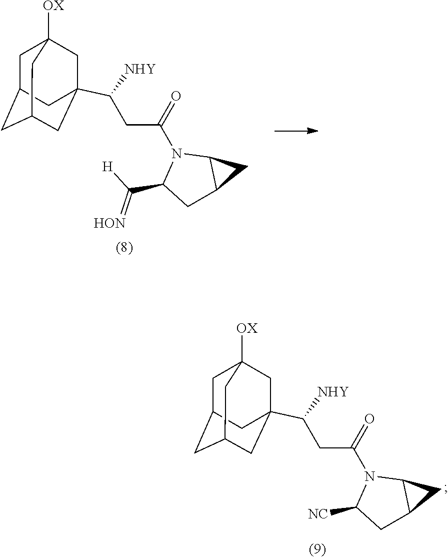

c) optional cleavage of the protecting groups X, Y to give saxagliptin (10):

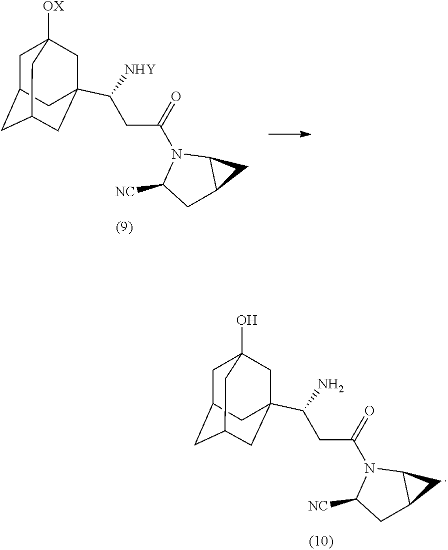

In the case of the synthesis of saxagliptin, a subsequent substitution by introducing the adamantyl fragment is more difficult in contrast to the production of vildagliptin. Therefore, the substituent is already present at the oxime stage. The substituents X and Y are each hydrogen or a protecting group. In particular, preference is given to readily cleavable acyl protecting groups such as tert-butyloxycarbonyl (Boc), acetyl or silyl, in particularly trimethylsilyl or —S(O₂)R, especially tosyl (CH₃—C₆H₄—SO₂—).

For the method conditions of the production of vildagliptin and saxagliptin or salts thereof, reference is made to the aforementioned description. Advantages arise in particular from the preparatively simple form and the mild conditions. This allows an economically viable synthesis of the gliptins. In addition, these can be achieved in high yield and enantiomeric excess.

The dehydration in step b) is carried out in each case preferably using a chemocatalyst, particularly in the presence of a transition metal catalyst, for example a Cu(II), Zn(II), Co(II) or Ni(II) catalyst. In this case, particular preference is given to Cu(II)-based chemocatalysts such as copper(II) acetate. Preferably, the mole fraction of the catalyst is in the range from ≥0.1 mol % to ≤25 mol %, preferably in the range from ≥1 mol % to ≤10 mol %, preferably in the range from ≥2 mol % to ≤5 mol %, based on the amount of substrate.

Preferably, the condensation of the aldehyde with hydroxylamine in step a) is carried out in aqueous solution, especially in a mixture of water and alcohol. Preferred alcohols are selected from the group comprising methanol, ethanol, isopropanol, n-propanol, n-butanol, tert-butanol, phenol and/or mixtures thereof. The alcohol is selected in particular from n-propanol and/or ethanol. Particularly suitable are mixtures of water and alcohol, for example mixtures of water with ethanol and/or n-propanol. For the dehydration of the aldoxime to give the nitrile in step b), organic solvents in particular can be used. The dehydration of the aldoxime to the α-aminonitrile is preferably carried out in a solvent selected from dichloromethane, methyl-tert-butyl ether, ethyl acetate, tetrahydrofuran, 2-methyltetrahydrofuran, toluene, acetonitrile, propionitrile, butyronitrile and/or mixtures thereof. In preferred embodiments, the dehydration of the aldoxime to give the α-aminonitrile is carried out in the presence of a nitrile component. The nitrile is preferably selected from the group comprising acetonitrile, propionitrile and/or butyronitrile. Particular preference is given to acetonitrile. The nitrile component is preferably present in the range of ≥10 eq., based on the aldoxime. The nitrile component may also be present at a higher proportion, for example ≥15 eq., based on the aldoxime. Furthermore, preference is given to mixtures of a nitrile, especially acetonitrile, with other solvents such as dichloromethane.

The condensation of the aldehyde with hydroxylamine in step a) can be carried out at ambient temperature. The dehydration of the aldoxime to give the nitrile in step b) is preferably carried out at elevated temperatures or under reflux. Preferably, the dehydration of the aldoxime to give the α-aminonitrile in step b) is conducted at a temperature in the range from ≥20° C. to ≤150° C., preferably in the range from ≥50° C. to ≤100° C., preferably in the range from ≥80° C. to ≤85° C. It can be envisaged that a reaction time of 1 to 7 or 8 hours at these temperatures is followed by a further reaction phase of up to 20 hours at ambient temperature.

Examples which serve to elucidate the present invention are specified below.

General Procedure

Chemicals and substances were purchased from Sigma-Aldrich or other commercial laboratory chemical providers and used without further purification.

Reversed-phase high-performance liquid chromatography (RP-HPLC) was performed on a Nucleodur C₁₈ Htec (Macherey-Nagel), using an eluent composed of water/acetonitrile 50:50 (v/v) under the following conditions: 1.0 mL/min, 40° C., 220 nm.

Normal phase high performance liquid chromatography (NP-HPLC) was performed on a Daicel Chiracel AD-H, using an eluent composed of CO₂/isopropanol 95:5 (v/v), under the following conditions: 0.75 mL/min, 30 min up to a ratio of 90:10, 2.0 mL/min, 30 min, 20° C., 210 nm.

Gas chromatography (GC) was performed on a Lipodex E (Macherey-Nagel) (0.25 mm ID×25 m length, 0.25 μm film) at 120° C. starting temperature (35 min), 20° C./min temperature ramp and 180° C. end temperature or on a CP-Chirasil-Dex CB (Agilent) (0.32 mm ID×25 m length, 0.25 μm film), 160° C. starting temperature (7 min), 2° C./min temperature ramp, 180° C. end temperature.

General Procedure for the Preparation of Chiral N-Acyl-α-Aminonitriles from Aldehydes

Step a) Condensation of the Aldehyde with Hydroxylamine:

Hydroxylamine hydrochloride (1.5 eq.) and sodium carbonate (1.5 eq.) were dissolved at room temperature (20±2° C.) in a mixture of water and n-propanol or water and ethanol. After addition of the aldehyde, the reaction mixture was stirred vigorously until TLC reaction monitoring (cyclohexane/ethyl acetate in various compositions) showed complete conversion. The reaction solution was extracted three times with ethyl acetate (1:1 v/v) and the combined organic phases were washed with water (1:3 v/v). After drying over MgSO₄, filtration and removal of the solvent, the crude product was obtained which was purified by column chromatography as required. The E/Z ratio of the product was determined by ¹H-NMR spectroscopy in CD₂Cl₂ or in CDCl₃.

Step b) Dehydration of the Aldoxime to Give a Chiral N-Acyl-α-Aminonitrile with Copper(II) Catalysis:

Copper(II) acetate (10 mol % or 2 mol %) was dissolved in acetonitrile. After addition of the aldoxime, the reaction mixture changed colour spontaneously from cyan to dark green. The suspension was heated to reflux for 60 min or 7 hours. After removal of the acetonitrile under reduced pressure, complete conversion was established by TLC analysis (cyclohexane/ethyl acetate in various compositions). The crude product, which comprised one equivalent of acetamide, was dissolved in cyclohexane/ethyl acetate (2:1 v/v) and filtered through a short silica gel column (4 cm) in order to remove acetamide and residual copper salts. After removal of the solvent, the desired nitrile was obtained. In order to determine the absolute configuration, the product was analyzed by chiral HPLC or chiral GC. Conversion to the nitrile was also determined by RP-HPLC or GC as an alternative to ¹H-NMR spectroscopy.

### EXAMPLE 1

### Preparation of (S)—N-Boc-pyrrolidinecarbonitrile

### 1a) Preparation of E/Z—N-Boc-1-proline Aldoxime

The synthesis was carried out analogously to the general procedure as described for step a). 104 mg of hydroxylamine hydrochloride (1.50 mmol) and 159 mg of sodium carbonate (1.50 mmol) were dissolved in 3 mL of water and 2 mL of ethanol at room temperature. After addition of 199 mg of N-Boc-1-prolinal (1.00 mmol), the solution was stirred at room temperature for 20 hours until the TLC reaction monitoring showed complete conversion. A colourless oil was obtained after work-up. The crude product was purified by column chromatography (cyclohexane/ethyl acetate 3:1, v/v). After removal of the solvent at 40° C. under reduced pressure, the product was obtained as a colourless oil with an E/Z ratio of 65:35. The E and Z isomers could not be separated. The isomers were confirmed by ¹H-NMR spectroscopy and GC. The yield of E/Z—N-Boc-1-proline aldoxime was 143 mg (67%).

### 1b) Preparation of (S)—N-Boc-pyrrolidinecarbonitrile

The synthesis was carried out analogously to the general procedure as described for step b). To a solution of 123 mg of E/Z—N-Boc-1-proline aldoxime (570 μmol) in 7 ml of acetonitrile were added 2.73 mg of copper(II) acetate (15.0 μmol). The reaction mixture was heated to reflux for 7 hours and then stirred at room temperature for 16 hours. After work-up, the product was obtained as a colourless oil with an enantiomeric excess of 97%. The yield of (S)—N-Boc-pyrrolidinecarbonitrile was 97 mg (86%).

### EXAMPLE 2

### Preparation of (R)—N-Boc-pyrrolidinecarbonitrile

### 2a) Preparation of E/Z—N-Boc-d-proline Aldoxime

The synthesis was carried out analogously to the general procedure as described for step a). 104 mg of hydroxylamine hydrochloride (1.5 mmol) and 159 mg of sodium carbonate (1.5 mmol) were dissolved in 3 mL of water and 2 mL of ethanol at room temperature. After addition of 199 mg of N-Boc-d-prolinal (1.0 mmol), the solution was stirred at room temperature for 24 hours until the TLC reaction monitoring showed complete conversion. A colourless oil was obtained after work-up. The crude product was purified by column chromatography (cyclohexane/ethyl acetate 2:1, v/v). After removal of the solvent at 40° C. under reduced pressure, the product was obtained as a colourless oil with an E/Z ratio of 72:28. The E and Z isomers could not be separated. The isomers were confirmed by ¹H-NMR spectroscopy and GC. The yield of E/Z—N-Boc-d-proline aldoxime was 177 mg (81%).

### 2b) Preparation of (R)—N-Boc-pyrrolidinecarbonitrile

The synthesis was carried out analogously to the general procedure as described for step b). To a solution of 161 mg of E/Z—N-Boc-d-proline aldoxime (750 μmol) in 7 ml of acetonitrile were added 2.73 mg of copper(II) acetate (15.0 μmol). The reaction mixture was heated to reflux for 7 hours and then stirred at room temperature for 16 hours. After work-up, the product was obtained as a colourless oil with an enantiomeric excess of 99%. The yield of (R)—N-Boc-pyrrolidinecarbonitrile was 130 mg (88%).

(R)—N-Boc-pyrrolidinecarbonitrile, which can be used as nitrile product in the synthesis of vildagliptin, was obtained with an enantiomeric excess of 99%. The synthesis therefore shows the robustness of the method to potential racemization.

### EXAMPLE 3

### Preparation of (R)—N-Boc-phenylalaninecarbonitrile

### 3a) Preparation of E/Z—N-Boc-d-phenylalanine Oxime

The synthesis was carried out analogously to the general procedure as described for step a). 146 mg of hydroxylamine hydrochloride (2.11 mmol) and 223 mg of sodium carbonate (2.11 mmol) were dissolved in 5 mL of water and 5 mL of 1-propanol at room temperature. After addition of 350 mg of N-Boc-d-phenylalaninal (1.40 mmol), the solution was stirred for 18 hours and complete conversion was confirmed by TLC monitoring. Work-up afforded a mixture of E/Z isomers of the product as a colourless solid.

The isomers were separated by column chromatography (cyclohexane/ethyl acetate 3:1, v/v), freed from solvent at room temperature and obtained as colorless solids. The isomers E-N-Boc-d-phenylalaninal oxime and Z—N-Boc-d-phenylalaninal oxime were confirmed by ¹H-NMR spectroscopy. The yield of E-N-Boc-d-phenylalaninal oxime was 200 mg (54%) and the yield of Z—N-Boc-d-phenylalaninal oxime was 142 mg (38%).

### 3b) Preparation of (R)—N-Boc-phenylalaninenitrile

The synthesis was carried out analogously to the general procedure as described for step b). 10.3 mg of copper(II) acetate (56.7 μmol) were suspended in 1.5 mL of acetonitrile. 150 mg of E/Z—N-Boc-d-phenylalaninal oxime (567 μmol) obtained in step a) was added and the reaction mixture was heated to reflux for 60 min. Work-up (cyclohexane/ethyl acetate 2:1, v/v) afforded the product as a colourless solid. In order to determine the absolute configuration, measurements were conducted by chiral HPLC. The retention time by RP-HPLC was Rₜ=9.0 min and the retention time by NP-HPLC was Rₜ=23.3 min. The reaction conversion was determined by RP-HPLC. The yield of (R)—N-Boc-phenylalaninenitrile was 116 mg (83%).

### EXAMPLE 4

### Preparation of (S)—N-Boc-phenylalaninenitrile

### 4a) Preparation of E/Z—N-Boc-1-phenylalanine Oxime

The synthesis was carried out analogously to the general procedure as described for step a). The synthesis was carried out according to SV1. 100 mg of hydroxylamine hydrochloride (1.43 mmol) and 152 mg of sodium carbonate (1.43 mmol) were dissolved in 5 mL of water and 5 mL of 1-propanol at room temperature. After addition of 238 mg of N-Boc-1-phenylalaninal (955 μmol), the solution was stirred for 18 hours and complete conversion was confirmed by TLC monitoring. Work-up afforded a mixture of E/Z isomers of the product as a colourless solid. The isomers were confirmed by ¹H-NMR spectroscopy. The yield of E/Z—N-Boc-1-phenylalanine oxime was 212 mg (84%).

### 4b) Preparation of (S)—N-Boc-phenylalaninenitrile

The synthesis was carried out analogously to the general procedure as described for step b). 7.3 mg of copper(II) acetate (40.2 μmol) were suspended in 1.0 mL of acetonitrile. 85.0 mg of E/Z—N-Boc-1-phenylalaninal oxime (322 μmol) obtained in step a) was added and the reaction mixture was heated to reflux for 60 min. Work-up (cyclohexane/ethyl acetate 2:1, v/v) afforded the product as a colourless solid. In order to determine the retention of the absolute configuration, measurements were conducted by chiral HPLC. The retention time by RP-HPLC was Rₜ=9.0 min and the retention time by NP-HPLC was Rₜ=20.9 min. The reaction conversion was determined by RP-HPLC. The yield of (S)—N-Boc-phenylalaninenitrile was 73 mg (92%).

### EXAMPLE 5

### Investigation of the Reaction Parameters of the Cu(II)-Catalyzed Synthesis of (S)—N-Boc-Pyrrolidinecarbonitrile

The Cu(II)-catalyzed synthesis of (S)—N-Boc-pyrrolidinecarbonitrile by dehydrating E/Z—N-Boc-1-proline aldoxime was carried out as has been described under example 1b) and the general procedure for step b), wherein the amount of acetonitrile and copper(II) acetate was in each case varied divergently or CH₂Cl₂ was added as co-solvent.

The results of the dehydrations are summarized in the following table:

TABLE 1

| | Amount of | Solvent | Amount of | Reaction | Conver- |
|---|---|---|---|---|---|
| Entry | CH₃CN | addition | Cu(OAc)₂ | time | sion |
| 1 | >100 equiv. | / | 10 mol % | 7 h at 80° | quanti- |
| | | | | C. + 16 h | tative |
| | | | | at 20° C. | |
| 2 | >100 equiv. | / | 2 mol % | 7 h at 80° | quanti- |
| | | | | C. + 16 h | tative |
| | | | | at 20° C. | |
| 3 | 10 equiv. | CH₂Cl₂ >25 | 2 mol % | 7 h at 80° | quanti- |
| | | equiv. | | C. + 16 h | tative |
| | | | | at 20° C. | |

As can be inferred from Table 1, complete conversion was achieved at a mole fraction of Cu(OAc)₂ as catalyst in a range from 2 to 10 mol %, based on the substrate. In addition, the amount of acetonitrile used could be reduced by using dichloromethane as co-solvent also with quantitative conversion.

1. Method for preparing an N-acyl- or N-sulfonyl-α-aminonitrile, comprising the following steps:
a) condensation of an N-acyl- or N-sulfonyl-α-aminoaldehyde with hydroxylamine to give an aldoxime, and
b) dehydration of the aldoxime obtained in step a) to give an N-acyl- or N-sulfonyl-α-aminonitrile.

2. The method according to claim 1, characterized in that in step a) an enantiomerically enriched or an enantiomerically pure N-acyl- or N-sulfonyl-α-aminoaldehyde is used, the absolute configuration of which is retained or substantially retained in the conversion to the N-acyl- or N-sulfonyl-α-aminonitrile.

3. The method according to claim 1, comprising the following steps:
a) condensation of an N-acyl- or N-sulfonyl-α-aminoaldehyde according to the general formula (I) with hydroxylamine to give an aldoxime according to the general formula (II), and
b) dehydration of the aldoxime obtained in step a) to give an N-acyl- or N-sulfonyl-α-aminonitrile according to the general formula (III): 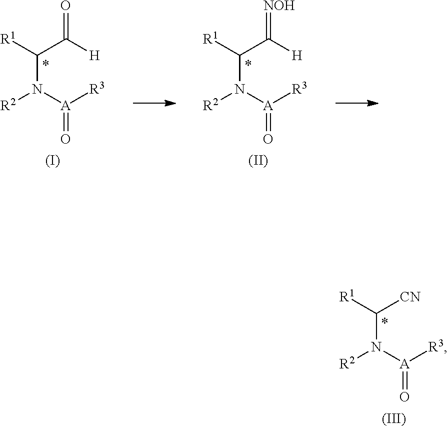
in which:
A is C or S═O;
R¹ is selected from the group comprising branched or unbranched C₁-C₂₀-alkyl, C₆-C₁₀-aryl, C₆-C₁₆-heteroaryl, C₇-C₁₆-arylalkyl and/or C₆-C₁₆-heteroarylalkyl, wherein these are unsubstituted or monosubstituted or polysubstituted by at least one substituent selected from the group comprising OH, NH₂, NHR⁴, NR⁴₂, C₁₋₄-alkyl, C₇-C₁₆-arylalkyl, C₆-C₁₆-heteroarylalkyl, carbonyl oxygen and/or C₁₋₄-alkoxy;
R² is selected from the group comprising H, branched or unbranched C₁-C₂₀-alkyl, C₆-C₁₀-aryl, C₆-C₁₆-heteroaryl, C₇-C₁₆-arylalkyl and/or C₆-C₁₆-heteroarylalkyl, wherein these are unsubstituted or monosubstituted or polysubstituted by at least one substituent selected from the group comprising OH, NH₂, NHR⁴, NR⁴₂, C₁₋₄-alkyl, C₇-C₁₆-arylalkyl, C₆-C₁₆-heteroarylalkyl, carbonyl oxygen and/or C₁₋₄-alkoxy; or
R¹ and R² together form a saturated 5- or 6-membered ring or a bicyclic ring system, wherein these may comprise at least one further heteroatom selected from N, O and/or S and/or these can be monosubstituted or polysubstituted by at least one substituent selected from the group comprising OH, NH₂, NHR⁴, NR⁴₂, C₁₋₄-alkyl, carbonyl oxygen and/or C₁₋₄-alkoxy;
R³ is selected from the group comprising H, C₁₋₆-alkoxy and/or C₁₋₆-alkyl, wherein these are monosubstituted or polysubstituted by at least one substituent selected from the group comprising OH, OR⁴, NH₂, NHR⁴, NR⁴₂, NHY and/or halogen; or is selected from the group of the structural elements (IV), (V) and (VI) as follows: 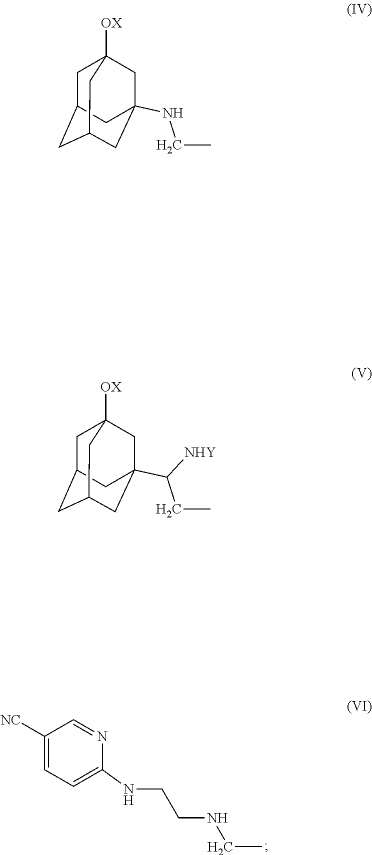
R⁴ is in each case identical or each independently selected from the group comprising C₁-C₁₈-alkyl or C₁-C₁₈-acyl;
X, Y are in each case identical or each independently H or a protecting group, especially selected from tert-butyloxycarbonyl (Boc), benzyloxycarbonyl, acetyl, silyl, p-tolyl, trifluoromethyl and/or sulfonyl.

4. The method according to claim 1, characterized in that the substituents R¹, R² and R³ of the compounds according to the general formulae (I), (II) and (III) are the following:
A is C;
R¹ is selected from the group comprising benzyl and/or C₁-C₂-alkyl,
R² is selected from the group comprising H, benzyl and/or C₁-C₂-alkyl, or
R¹ and R² together form a saturated 5-membered ring or bicyclo[3.1.0]hexane, and
R³ is selected from the group comprising H, tert-butoxy, chloromethyl, structural element (IV), (V) and/or (VI).

5. The method according to claim 1, characterized in that in step b) the dehydration of the aldoxime to give the N-acyl- or N-sulfonyl-α-aminonitrile is carried out in the presence of a transition metal catalyst, especially a Cu(II), Zn(II), Co(II) or Ni(II) catalyst.

6. The method according to claim 1, characterized in that the mole fraction of the catalyst is in the range from ≥0.1 mol % to ≤25 mol %, preferably in the range from ≥1 mol % to ≤10 mol %, preferably in the range from ≥2 mol % to ≤5 mol %.

7. The method according to claim 1, characterized in that the dehydration of the aldoxime to give the N-acyl- or N-sulfonyl-α-aminonitrile in step b) is carried out in the presence of a nitrile component preferably selected from the group comprising acetonitrile, propionitrile and/or butyronitrile, wherein the nitrile component is preferably present in the range of ≥10 eq., based on the aldoxime.

8. The method according to claim 1, characterized in that the dehydration of the aldoxime to give the N-acyl- or N-sulfonyl-α-aminonitrile in step b) is conducted at a temperature in the range from ≥20° C. to ≤150° C., preferably in the range from ≥50° C. to ≤100° C., preferably in the range from ≥80° C. to ≤85° C.

9. Method for preparing vildagliptin or salts thereof, comprising the following steps:
a) condensing an aldehyde of the formula (1) with hydroxylamine to give an aldoxime of the formula (2): 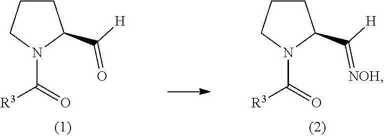
in which
R³ is —CH₂— substituted by a substituent selected from the group comprising OH, OR⁴, NH₂, NHR⁴, NR⁴₂, NHY and/or halogen or structural element (IV) as follows: 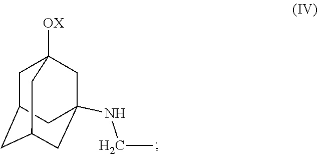
X, Y are identical or each independently H or a protecting group, especially selected from tert-butyloxycarbonyl (Boc), benzyloxycarbonyl, acetyl, silyl, p-tolyl, trifluoromethyl and/or sulfonyl:
R⁴ is identical or each independently selected from the group comprising C₁-C₁₈-alkyl or C₁-C₁₈-acyl;
b) dehydration of the aldoxime of the formula (2) obtained in step a) to give an N-acyl-α-aminonitrile of the formula (3): 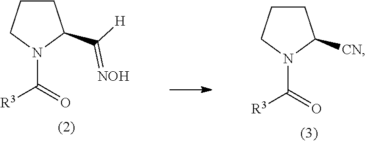
c) optional reaction of the N-acyl-α-aminonitrile of the formula (3), where R³ is —CH₂— substituted by a substituent selected from the group comprising OH, OR⁴, NH₂, NHR⁴, NR⁴₂, NHY and/or halogen, with 1-aminoadamantane-3-ol or a protected derivative of the formula (4) to give the compound of the formula (5): 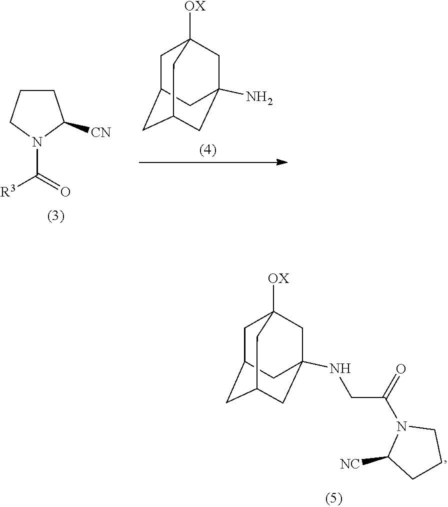
and
d) optional cleavage of the protecting group X to give vildagliptin of the formula (6):

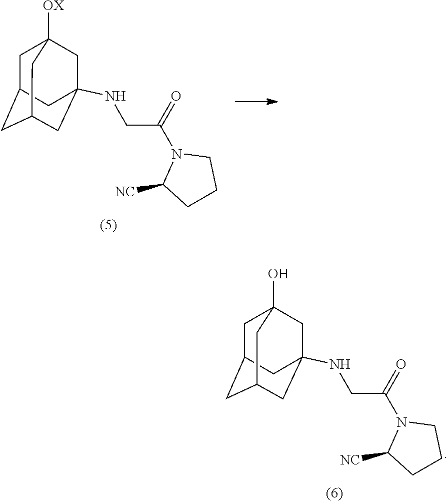

10. Method for preparing saxagliptin or salts thereof, comprising the following steps:
a) condensation of an aldehyde of the formula (7) with hydroxylamine to give an aldoxime of the formula (8): 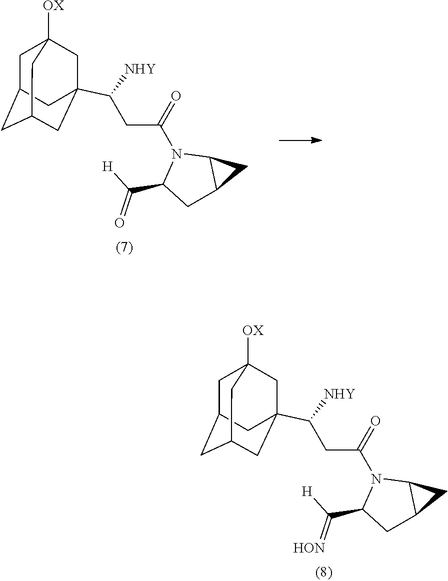
in which:
X, Y are identical or each independently H or a protecting group, especially selected from tert-butyloxycarbonyl (Boc), benzyloxycarbonyl, acetyl, silyl, p-tolyl, trifluoromethyl and/or sulfonyl;
b) dehydration of the aldoxime of the formula (8) obtained in step a) to give an N-acyl-α-aminonitrile of the formula (9): 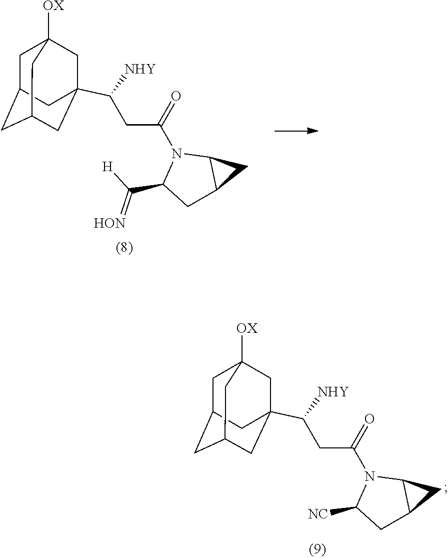
c) optional cleavage of the protecting groups X, Y to give saxagliptin (10): [chemical structure]
